(19) United States
(12) Patent Application Publication
(10) Pub. No.: US 2013/0204215 A1
(43) Pub. Date: Aug. 8, 2013

(54) Valve for Regulating the Flow of A Liquid

(71) Applicant: BIOFLO, LLC, Sanford, FL (US)

(72) Inventor: Kevin J. Spolski, Sanford, FL (US)

(73) Assignee: BIOFLO, LLC, Sanford, FL (US)

(21) Appl. No.: 13/837,926

(22) Filed: Mar. 15, 2013

Related U.S. Application Data
(63) Continuation-in-part of application No. 13/334,605, filed on Dec. 22, 2011.
Application No. 13/334,605 is a continuation-in-part of application No. 13/105,640, filed on May 11, 2011.

(51) Int. Cl. A61F 5/44

(52) U.S. Cl. CPC A61F 5/4405
USPC 604/328

(57) ABSTRACT

A new valve for regulating the flow of liquid has two housings that are removably attached to one another. When the housings are separated, the liquid can no longer pass through the valve. The valve is typically connected to an indwelling bladder catheter and the valve allows the patient to be at least temporarily disconnected from a collection bag.

### BACKGROUND OF THE INVENTION

This application is a continuation-in-part of U.S. patent application Ser. No. 13/334,605, filed on Dec. 22, 2011, which is a continuation-in-part application U.S. application Ser. No. 13/105,640, filed on May 11, 2011, the contents of those applications being incorporated herein in their entireties by reference.

### FIELD OF THE INVENTION

A new design for a valve that regulates the flow of a liquid has the ability to passively regulate the flow of liquid and allows the valve housing to be disconnected, allowing movement of the person to whom the valve is connected away from a collection bag.

Many people, at home, in a hospital, and in third-party care facilities require the use of an in-dwelling bladder catheter because of a medical condition. However, being constantly attached to a bladder bag that holds the liquid (urine) is inconvenient, and potentially and unnecessarily limits the person's movement and living conditions.

A new valve allows for the cyclical emptying of the bladder and allows the patient to be disconnected from the collection bag.

### SUMMARY OF THE INVENTION

The present invention is directed to a valve for regulating the flow of a liquid therethrough that includes a first housing having an inlet and an outlet, the first housing having a first magnet housing disposed therein, the first magnet housing having a base, the base having a first side and a second side and at least two extensions extending from the first side, the at least two extensions engaging an inside surface of the first housing and retaining a magnet between the first magnet housing and the first housing, a second housing removably attachable to the first housing, the second housing having an opening extending therethrough, and a sealing member disposed in the first housing adjacent the outlet, the sealing member sealing the outlet in the first housing when the second housing is disengaged from the first housing.

In some embodiments, a sampling port is disposed in the first housing and wherein the at least two extensions are disposed around an opening in the base and have an angle between them, the angle being 180 degrees or less, the at least two extensions being disposed in the first housing such the sampling port bisects the angle between the at least two extensions when the first magnet housing is disposed within the first housing.

In another aspect, the invention is directed to a valve for regulating the flow of a liquid therethrough that includes a first housing having an inlet and an outlet, the first housing having a first magnet housing disposed therein, the first magnet housing having a base, the base having a first side and a second side and at least two extensions extending from the first side, the at least two extensions engaging an inside surface of the first housing and retaining a first magnet between the first magnet housing and the first housing, a second housing removably attachable to the first housing, the second housing having an opening extending therethrough, a second magnet disposed in the first housing, the second magnet movable relative to the first magnet, and a sealing member disposed in the first housing adjacent the outlet, the sealing member sealing the outlet in the first housing when the second housing is disengaged from the first housing.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description of the present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is a front perspective view of one embodiment of a valve for regulating the flow of a liquid according to the present invention;

FIG. 2 is a front perspective view of the housings of the valve in FIG. 1 separated from one another;

FIG. 3 is a rear perspective view of the housings of the valve in FIG. 1 separated from one another;

FIG. 4 is an exploded perspective view of the valve of FIG. 1;

FIG. 5 is a cross sectional view from the left side of the valve in FIG. 4;

FIG. 6 is a cross section view of the valve along the line 6-6- in FIG. 1 with one magnet in a first position and sealing the valve;

FIG. 7 is a cross section view of the valve along the line 7-7 in FIG. 1;

FIG. 8 is a cross section view of the valve along the line 8-8 in FIG. 1 with the magnet in a second position and allowing liquid to flow therethrough;

FIG. 9 is a cross section view of the valve along the line 9-9 in FIG. 2;

FIG. 10 is a perspective view of the valve connected to tubing on one end and a collection bag on the other end;

FIG. 11 is an exploded perspective view of another embodiment of a valve for regulating the flow of a liquid according to the present invention;

FIG. 12 is a cross sectional view of the left side of the valve in FIG. 11;

FIG. 13 is a cross sectional view of the left side of the valve in FIG. 11 in an assembled state;

FIG. 14 is a cross sectional view from the top of the valve in FIG. 11 in an assembled state;

FIG. 15 is a perspective view of the first magnet holder used in the valve in FIG. 11;

FIG. 16 is a cross sectional view of the left side of the valve in FIG. 11 with the housings separated;

FIG. 17 is a cross sectional view through the first magnet housing toward the inlet in the valve in FIG. 11;

FIG. 18 is a cross sectional view of a portion of the first housing of the valve in FIG. 11;

FIG. 19 is an exploded perspective view of another embodiment of a valve for regulating the flow of a liquid according to the present invention;

FIG. 20 is a cross sectional view of the left side of the valve in FIG. 19;

FIG. 21 is a cross sectional view of the left side of the valve in FIG. 19 in an assembled state;

FIG. 22 is a cross sectional view of the left side of the valve in FIG. 19 with the housings separated;

FIG. 23 is a partial cross sectional view of a portion of the valve showing the elastic element;

FIG. 24 is a front perspective view of the sealing member; and

FIG. 25 is end view through the elastic member engaging the sealing member.

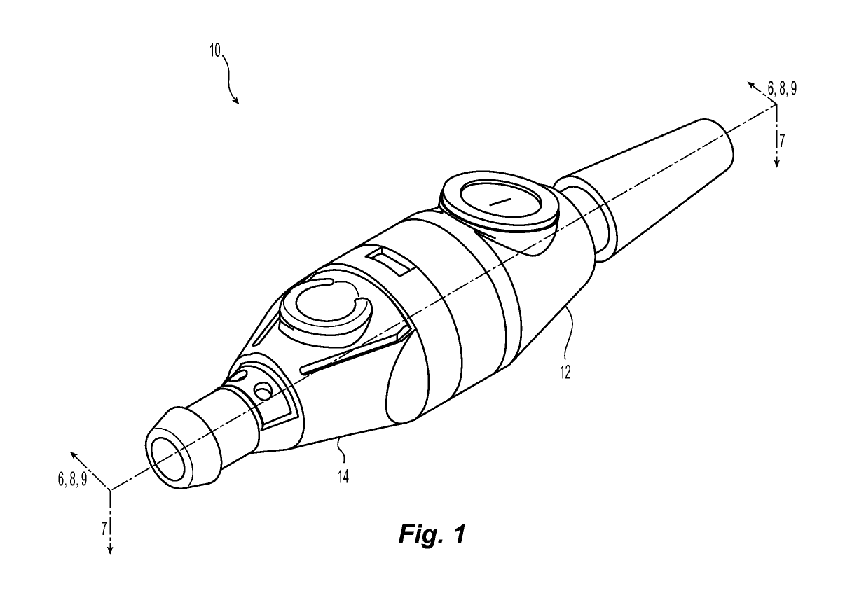

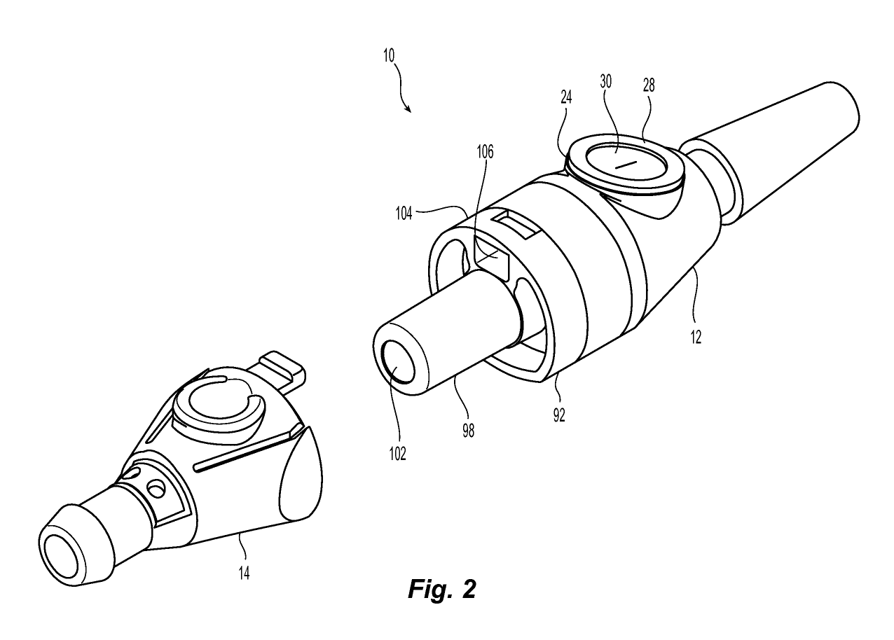

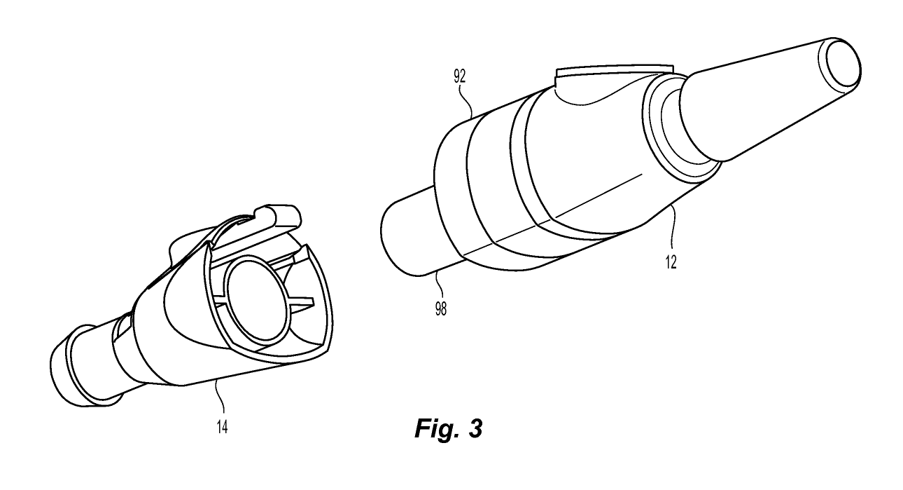

### DETAILED DESCRIPTION OF THE PREFERRED EMBODIMENTS

Reference will now be made in detail to the present preferred embodiment(s) of the invention, examples of which are illustrated in the accompanying drawings. Whenever possible, the same reference numerals will be used throughout the drawings to refer to the same or like parts.

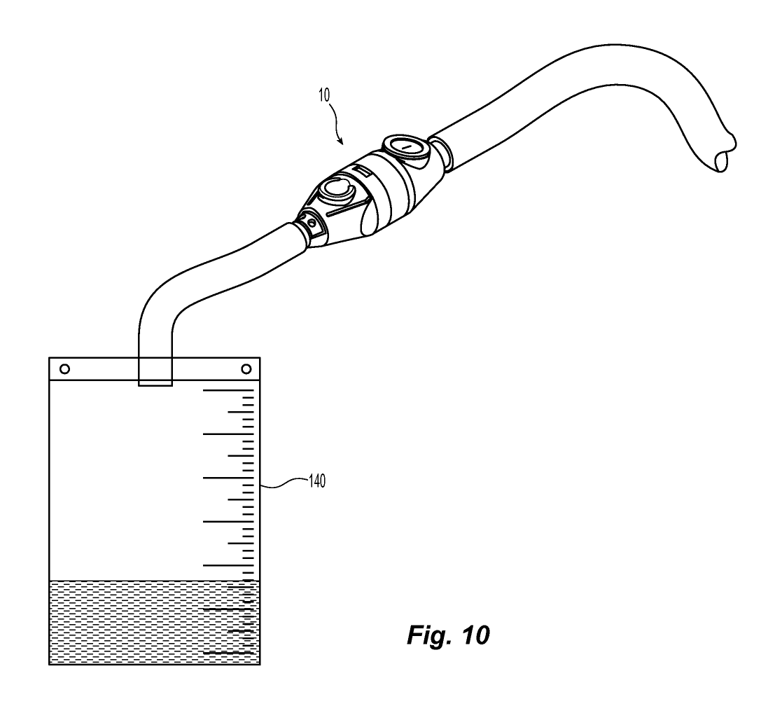

One embodiment of the present invention illustrated in the figures is directed is a valve 10 for regulating the flow of a liquid. The valve 10 has a first housing 12 and a second housing 14 that is removably attachable to the first housing 12. The first housing 12 has a catheter connector 16 having a proximal end 18 and a distal end 20, the proximal end 18 of the catheter connector 16 has an inlet 22 that is configured to engage a tubing (see, e.g., FIG. 10). The inlet 22, as illustrated, has a generally smooth outer surface that increases in diameter from the end of the inlet 22 toward the remaining portion of the catheter connector 16. The inlet 22 may have any other configuration that allows for connection to tubing and still fall within the scope of the present invention. The first and second housings 12,14 are preferably made from a K-Resin SBC material.

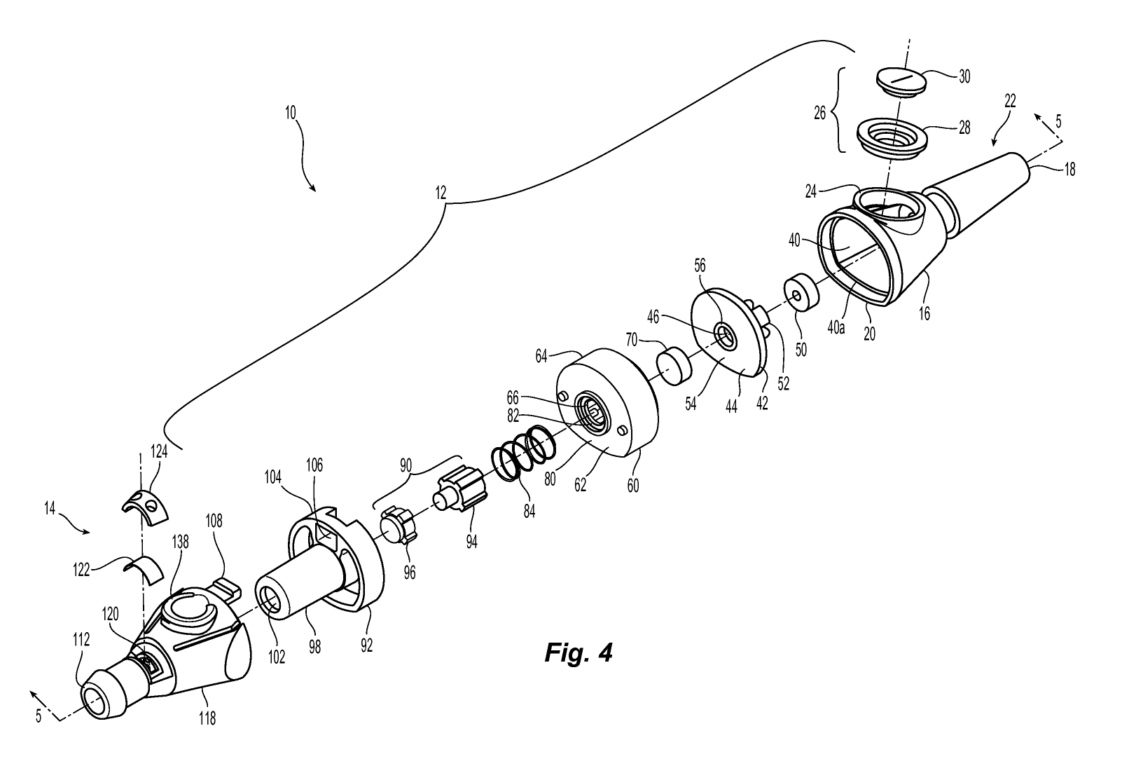

As best illustrated in FIG. 4, the catheter connector 16 also has an opening 24 that functions as a sampling port. The opening 24 preferably has a needleless port 26. The needleless port 26 allows for insertion of a needleless syringe (not shown) to withdraw a sample of the liquid (generally urine) for testing. The needleless port 26 is preferably a resealable opening. The needleless port 26 has a main body 28 and the resealable portion 30, the resealable portion 30 may or may not have a pre-cut slit therein. It is also possible that the needleless port 26 is a single unit and not made of two different portions. The main body 28 is preferably made of polypropylene and the resealable portion 30 is preferably made of a thermoplastic elastomer, but any appropriate materials may be used.

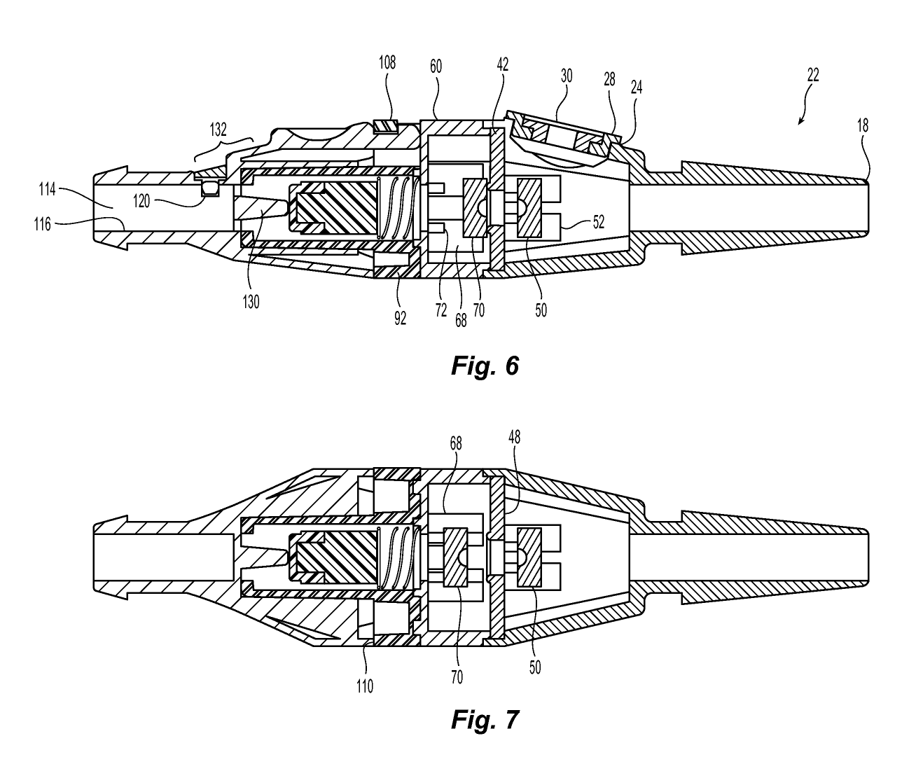

The catheter connector 16 also has an opening 40 at the distal end 20 that is in fluid communication with the inlet 22. The opening 40 has a recessed portion 40a adjacent the distal end 20 into which a first magnet housing 42 is disposed, sealing the opening 40 of the catheter connector 16. See, e.g., FIGS. 6-9. The first magnet housing 42 has a base plate 44 that has the same configuration as the opening 40 and an opening 46 in the base plate 44 to allow the liquid to pass therethrough. On a first side 48 of the base plate 44 a first magnet 50 is secured. The first magnet 50 is secured in extensions 52 that extend from the first side 48 of the base plate 44 to keep the first magnet 50 at a predetermined distance from the opening 46 and a second magnet, which is described in more detail below. The first magnet housing 42 preferably has on a second side 54 a raised portion 56 around the opening 46. As described in more detail below, the raised portion provides a surface against which the second magnet can maintain contact to seal the opening 46.

As best illustrated in FIGS. 4 and 6-9, second magnet housing 60, also a part of the first housing 12, is attached to the first magnet housing 42 and the catheter connector 16 and extends distally from the catheter connector 16. The second magnet housing 60 is preferably generally cup-shaped, having a base member 62 and a peripheral wall 64, with an opening 66 in the base member 62 to allow the liquid to flow therethrough. Extending from the base member 62 toward the catheter connector 16 and the first magnet housing 40 are extensions 68 that slidingly hold a the second magnet 70. Preferably, there are four extensions 68, but there may be other numbers of extensions and still be within the scope of the invention. The extensions 68 also have a surface 72 to engage the second magnet 70 and prevent the second magnet 70 from moving too far distally (toward the base member 62). The second magnet 70 is drawn magnetically toward the first magnet 50 causing the second magnet 70 to engage the raised portion 56 around the opening 46, thereby closing the opening 46 and preventing the flow of liquid through the valve 10. However, when sufficient liquid is present in the opening 40 and exerts pressure on the second magnet 70 sufficient to overcome the magnetic attraction between the two magnets 50,70, then the second magnet 70 moves axially away from the opening 46 within the extensions 68 (but no farther than the surfaces 72) to allow the liquid to drain through the opening 46 (and the tubing that is inserted into bladder of a patient). When the liquid has drained away and removes this force, then the magnetic attraction causes the second magnet 70 to once again close the opening 46.

The second magnet housing 60 has on a bottom side 80 a recessed portion 82 to receive an elastic member 84 that engages and biases a sealing member 90 toward the second housing 14 in a drain end 92. While a coil spring is illustrated as the elastic member 84, any appropriate style of spring or elastic member may be used to bias the sealing member 90. The sealing member 90 preferably has two elements, a main sealing member 94 and a resilient cover member 96 that is attached to the main sealing member 94. However, the sealing member 90 may be one integral element rather than two separate elements and may be made from a single material rather than multiple materials.

The drain end 92, which constitutes the last element of the first housing 12, is attached to the second magnet housing 60 and is in fluid communication with the inlet 22 in the catheter connector 16. See FIGS. 4 and 6-9. The drain end 92 has a generally cylindrical center portion 98 in which the sealing member 90 is slidingly movable. The cylindrical center portion 98 has an inlet 100 and an outlet 102. The sealing member 90 is, as noted above, biased away from the second magnet housing 60 and toward the outlet 102 of the drain end 92. The drain end 92 also has an outer portion 104 that is secured to the bottom side 80 of the second magnet housing 60. Between the cylindrical center portion 98 and the outer portion 104 is an opening 106 into which a cantilevered latch 108 from the second housing 14 is inserted to hold the second housing 14 to the first housing 12.

The second housing 14 has a proximal end 110 and a distal end 112. The second housing 14 has an inner opening 114 that extends between the proximal end 110 and the distal end 112 and defines an inner surface 116. The second housing 14 also has a outside surface 118 and an opening 120 that extends between the outside surface 118 and the inner surface 116. The opening 120 functions as a vent to allow air to enter the valve 10 and the liquid to move through the valve 10 and into the collection bag 140. See FIG. 10. The opening 120 is preferably covered by a Tyvek covering 122 and a cover 124, which allows air to enter into the valve 10 for complete (or near-complete) emptying of the valve 10 without allowing the liquid to escape therethrough.

Positioned within the inner opening 114 is a projection 130 that extends from central portion 132 of the inner opening 114 toward the proximal end 110. The inner opening 114 also has in the central portion 132 (and preferably at the location of the distal end 134 of the projection 130) a proximally-facing surface 136. The inner opening 114 is configured and sized to receive the cylindrical center portion 98 of the first housing 12 therein and the proximally-facing surface 136 is positioned and configured such that the outer portions of outlet 102 of the drain end 92 make contact with the surface 136 and seal the valve 10 to prevent leaking when the two housings 12,14 are attached. The projection 130 is configured and sized, when the second housing 14 is connected to the first housing 12, to engage the sealing member 90 through the outlet 102 of the drain end 92 and move it axially and in a proximate direction (toward the inlet 22). This allows for the liquid to drain from the first housing 12 in to the second housing 14. Openings spaced around the bottom of the projection 130 allow the liquid to pass around the projection 130 and along the inner surface 114. Since the proximal end 110 of the second housing 14 is, by way of the latch 108, removably mounted to the first housing 12, removing the second housing 14 also removes the projection 130 from the drain end 92, allowing the sealing member 90 to seal the outlet 102, even if the second magnet 70 moves to allow the liquid to pass through the opening 46. This allows the patient to, at least temporarily, be separated from a collection bag 140, which is illustrated in FIG. 10.

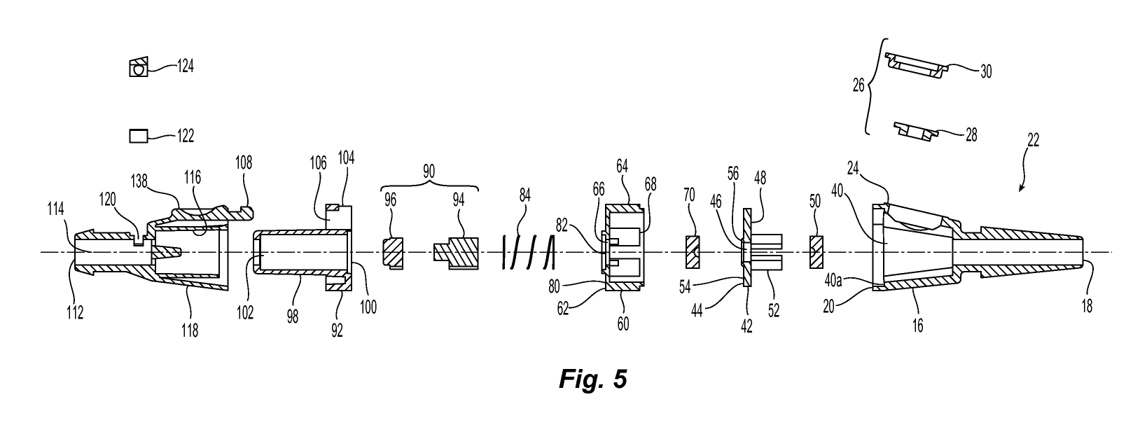

As noted above, the first and second housings 12,14 are removably attached to one another by the latch 108. By “removably attached,” Applicant means that the two housings 12,14 are intended to and can repeatedly engage and disengage one another without any other elements (e.g., glues, adhesives, bands, etc.), structures, or destroying any portions or parts that are intended to be used to attach housings 12,14. As best seen in FIGS. 5 and 6, the latch 108 is integral with the button 138, which when pressed then causes the latch 108 to be disengaged from the outer portion 104 of the drain end 92, and the housing 14 can be removed from housing 12. It should also be noted that due to the tight fit of the cylindrical center portion 98 in the inner opening 114, only one latch 108 needs to be used to maintain the connection between the two housings 12,14. Other types of latches and numbers of latches may also be used with the valve 10 and still come within the scope of the present invention.

Turning now to FIGS. 6-10, the operation of the valve 10 will be described in more detail. FIG. 6 is a cross sectional view of the valve 10 through the latch 108. The position of the second magnet 70 is in the proximal position, that is the second magnet 70 is sealing off the opening 46 as it makes contact with the raised portion 56 around the opening 46. As can be seen in the left side of the figure, the projection 130 has engaged the sealing member 90 through the outlet 102 since the second housing 14 is attached to the first housing 12.

FIG. 7 is a cross sectional view of the valve 10 at a 90° angle to the view in FIG. 6. In this figure, the second magnet 70 is illustrated as if the liquid has exerted a sufficient force on the second magnet 70 to move it away from the raised portion 56 around the opening 46 and toward the surfaces 72.

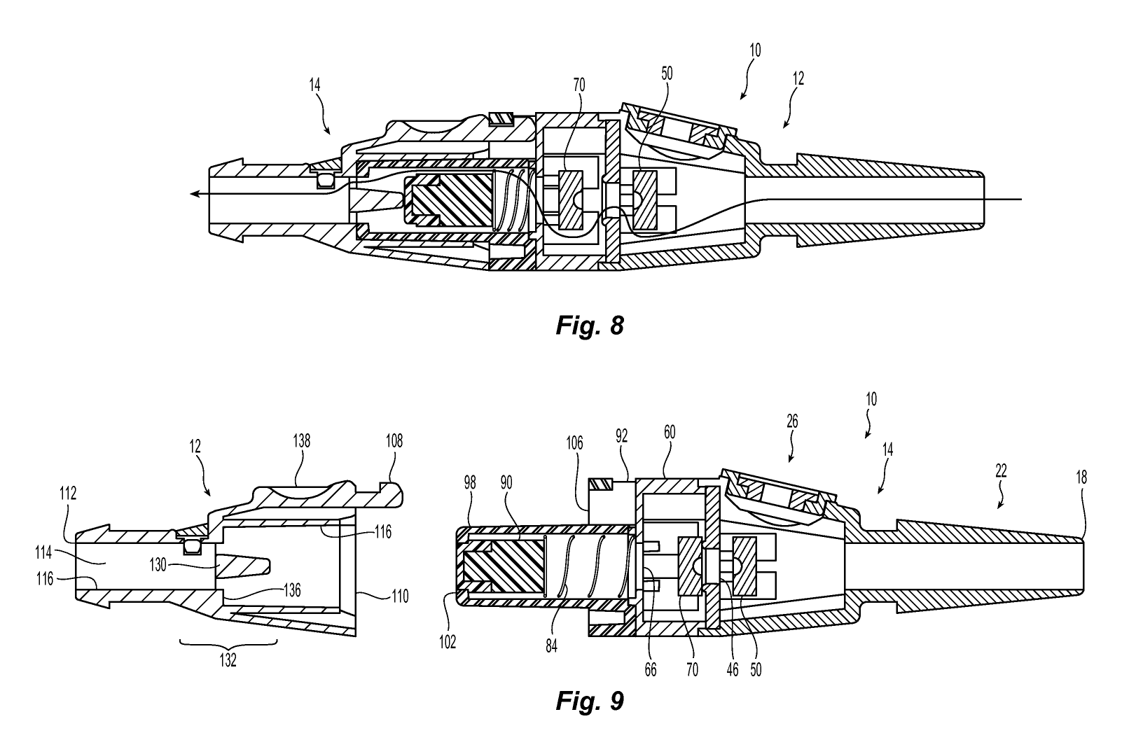

FIG. 8 is a cross sectional view of valve 10 in the same orientation as FIG. 6, but the second magnet 70 is the open position and not closed as in FIG. 6. Additionally, the arrow indicates at least one path for the liquid to pass through the valve 10.

FIG. 9 is a cross section of the valve 10 with the first housing 12 and the second housing 14 separated from one another. In this figure, it is clear that the projection 130 no longer engages the sealing member 90, and the sealing member 90 is firmly in the outlet 102, preventing the liquid from exiting the first housing 12. This configuration allows a patient to disconnect the housings 12,14 for better mobility (or other reasons), and not have to worry about the liquid draining onto the floor or other equally distasteful places. While the second magnet 70 is illustrated as being against the raised portion 56 around the opening 46, thereby closing the opening 46, even if opening 46 were open, the liquid still would not leak from the first housing 12.

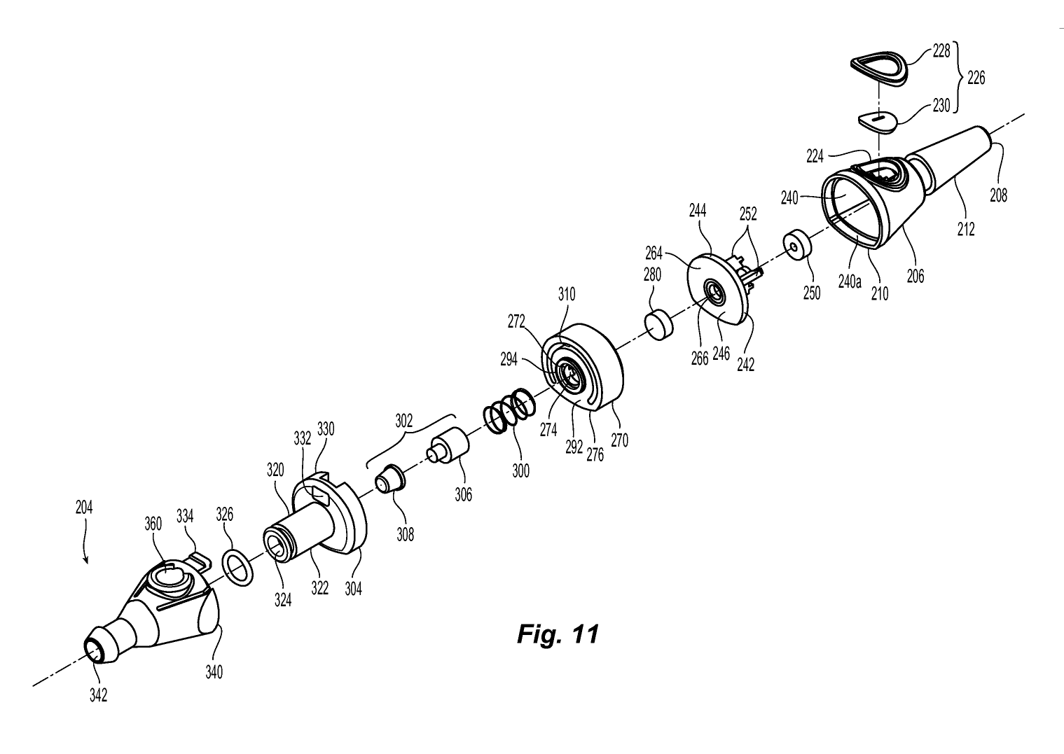

Another embodiment of a valve 200 according to the present invention is illustrated in FIGS. 11-18. The valve 200 has a first housing 202 and a second housing 204 that is removably attachable to the first housing 202. The first housing 202 has a catheter connector 206 having a proximal end 208 and a distal end 210, the proximal end 208 of the catheter connector 206 has an inlet 212 that is configured to engage a tubing (see, e.g., FIG. 10). The inlet 212, as illustrated, has a generally smooth outer surface that increases in diameter from the end of the inlet 212 toward the remaining portion of the catheter connector 206. The inlet 212 may have any other configuration that allows for connection to tubing and still fall within the scope of the present invention. The first and second housings 202,204 are preferably made from a polycarbonate.

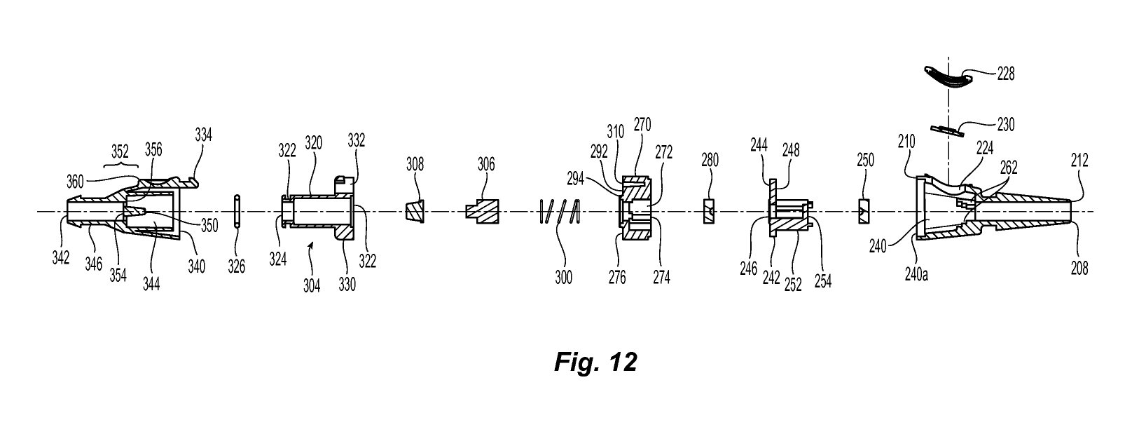

As best illustrated in FIGS. 11 and 12, the catheter connector 206 also has an opening 224 that functions as a sampling port. The opening 224 preferably has a needleless port 226. The needleless port 226 allows for insertion of a needleless syringe (not shown) to withdraw a sample of the liquid (generally urine) for testing. The needleless port 226 is preferably a resealable opening. The needleless port 226 has a main body 228 and the resealable portion 230, the resealable portion 230 may or may not have a pre-cut slit therein. It is also possible that the needleless port 226 is a single unit and not made of two different portions. The main body 228 is preferably made of polypropylene and the resealable portion 230 is preferably made of Dynaflex® thermoplastic elastomer (available from GLS Corp.), but any appropriate materials may be used. Additionally, a syringe with a needle may also be used with the needleless port 226, as discussed in more detail below regarding the configuration of the needleless port 226 to the internal structures.

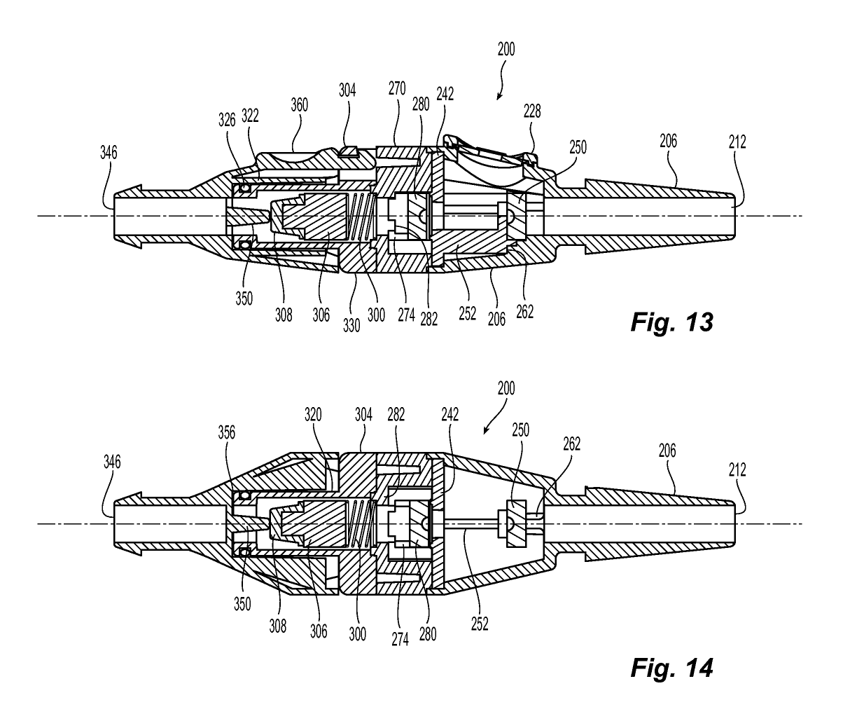

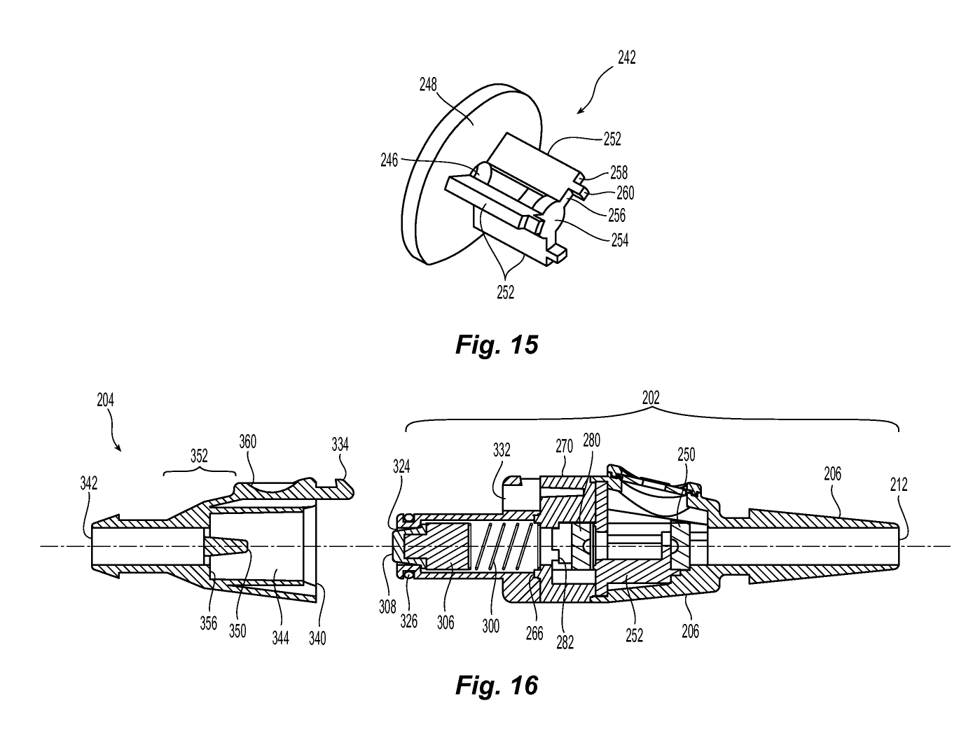

The catheter connector 206 also has an opening 240 at the distal end 220 that is in fluid communication with the inlet 212. The opening 240 has a recessed portion 240a adjacent the distal end 210 into which a first magnet housing 242 is disposed, sealing the opening 240 of the catheter connector 206. See, e.g., FIGS. 13 and 14. The first magnet housing 242 is preferably a monolithic element that is injection molded as a single element from Dynaflex® thermoplastic elastomer. However, the various parts of the first magnetic housing 242 may also be assembled and attached to one another by appropriate means for the materials (e.g., adhesives, ultrasonic welding, etc.). As best seen in FIG. 15, first magnetic housing 242 has a base plate 244 that has the same configuration as the opening 240 and an opening 246 in the base plate 244 to allow the liquid to pass therethrough. On a first side 248 of the base plate 244 a first magnet 250 is secured. The first magnet 250 is secured in between extensions 252 and on a support member 254. The extensions 252 extend from the first side 248 of the base plate 244, and in conjunction with the support member 254, keep the first magnet 250 in a predetermined relationship with the opening 246 and a second magnet, which is described in more detail below. The support member 254 also provides support and stability to the extensions 252. The top of the extensions 252 have a stepped configuration that serves two purposes. First, the extensions 252 have an inner step 256 that preferably engages the first magnet 250 to support and contain the first magnet 250 relative to the inlet 212 and the opening 240. The second purpose of the outer step 258 and the top surface 260 of the extensions 252 is to engage the inner surface 262 of the catheter connector 206. As best illustrated in FIGS. 11, 13 and 18, the catheter connector 206 has a similar stepped configuration 262 that aligns with and engages the steps 256,258. When the first magnet housing 242 is inserted into the catheter connector 206 with the magnet 250 attached thereto, the steps 256,258 engage and may even compress slightly as the valve 200 is assembled.

The first magnet housing 242 also preferably has on a second side 264 a raised portion 266 around the opening 246. As described in more detail below, the raised portion 266 provides a surface against which the second magnet can maintain contact to seal the opening 246.

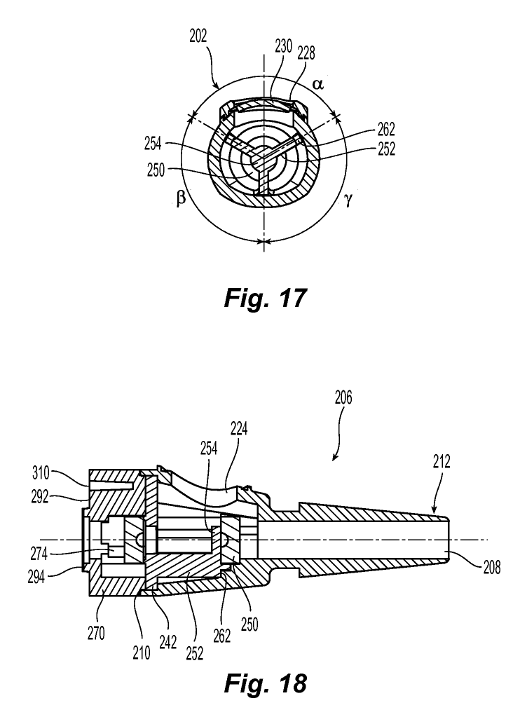

As illustrated in FIG. 17, the extensions 252 are preferably disposed evenly around the opening 246 in the first magnet housing 242. As illustrated, the extensions 252 have an angle between them, including α, β, and γ. In FIG. 17, the angles are all the same (α=β=γ), but they could have different values. Additionally, a line A that is orthogonal to the valve 200 and passes through the center of the valve 200 bisects the opening 224 and needleless port 226. Line A also preferably bisects the angle formed by two adjacent extensions 252 that are disposed closest to the opening 224. In this way, if the user or a healthcare worker were to use a syringe with a needle to take a sample, the needle would have a substantial amount of space in which to retrieve the sample and not be inserted into the extensions.

As best illustrated in FIGS. 11, 13, and 14, second magnet housing 270, also a part of the first housing 202, is attached to the first magnet housing 242 and the catheter connector 206 and extends distally from the catheter connector 206. The second magnet housing 270 is preferably generally cup-shaped with a central opening 272 to allow the liquid to flow therethrough. The central opening 272 has extensions 274 that extend from the distal end 276 toward the catheter connector 206 and the first magnet housing 240 that slidingly hold a the second magnet 280. Preferably, there are three extensions 274, but there may be other numbers of extensions and still be within the scope of the invention. The extensions 274 also have a surface 282 to engage the second magnet 280 and prevent the second magnet 280 from moving too far distally (toward the distal end 276). The second magnet 280 is drawn magnetically toward the first magnet 250 causing the second magnet 280 to engage the raised portion 266 around the opening 246, thereby closing the opening 246 and preventing the flow of liquid through the valve 200. However, when sufficient liquid is present in the opening 240 and exerts pressure on the second magnet 280 sufficient to overcome the magnetic attraction between the two magnets 250,280, then the second magnet 280 moves (is pushed) axially away from the opening 246 within the extensions 274 (but no farther than the surfaces 282) to allow the liquid to drain through the opening 246 (and the tubing that is inserted into bladder of a patient). When the liquid has drained away and removes this force, then the magnetic attraction causes the second magnet 280 to once again close the opening 246.

The second magnet housing 270 has on a bottom side 292 an elevated ring portion 294 that engages and centrally maintains an elastic member 300, which in turn, engages and biases a sealing member 302 toward the second housing 204 in a drain end 304. While a coil spring is illustrated as the elastic member 300, any appropriate style of spring or elastic member may be used to bias the sealing member 302. The sealing member 302 preferably has two elements, a main sealing member 306 and a resilient cover member 308 that is attached to the main sealing member 302. However, the sealing member 302 may be one integral element rather than two separate elements and may be made from a single material rather than multiple materials. The second magnet housing 270 also has a recessed portion 310. The recessed portion 310, in this embodiment, is simply to reduce the volume and thereby the weight of the second magnet housing 270 as it is machined from a single block of material. The recessed portion 310 plays no other role in the valve 200.

The drain end 304, which constitutes the last element of the first housing 202, is attached to the second magnet housing 270 and is in fluid communication with the inlet 212 in the catheter connector 206. See FIGS. 13-14 and 16. The drain end 304 has a generally cylindrical center portion 320 in which the sealing member 302 is slidingly movable. The cylindrical center portion 320 has an inlet 322 and an outlet 324. The sealing member 302 is, as noted above, biased away from the second magnet housing 270 and toward the outlet 324 of the drain end 304. The drain end 304 also has an outer portion 330 that is secured to the bottom side 292 of the second magnet housing 270. Between the cylindrical center portion 320 and the outer portion 330 is an opening 332 into which a cantilevered latch 334 from the second housing 204 is inserted to hold the second housing 204 to the first housing 202.

The cylindrical center portion 320 has a groove 322 near the outlet 324 and preferably adjacent the outlet 324. The location of the groove 324 need only be such that an elastic element 326, such as an o-ring, can engage the second housing 204 as described in more detail below.

The second housing 204 has a proximal end 340 and a distal end 342. The second housing 204 has an inner opening 344 that extends between the proximal end 340 and the distal end 342 and defines an inner surface 346.

Positioned within the inner opening 344 is a projection 350 that extends from central portion 352 of the inner opening 344 toward the proximal end 340. The inner opening 344 also has in the central portion 352 (and preferably at the location of the distal end 354 of the projection 350) a proximally-facing surface 356. The inner opening 344 is configured and sized to receive the cylindrical center portion 320 of the first housing 202 therein and the proximally-facing surface 356 is positioned and configured such that the outer portions of outlet 324 of the drain end 304 make contact with the proximally-facing surface 356 (and in particular the elastic element 326 in the groove 322) and seal the valve 200 to prevent leaking when the two housings 202,204 are attached. The projection 350 is configured and sized, when the second housing 204 is connected to the first housing 202, to engage the sealing member 302 through the outlet 324 of the drain end 304 and move it axially and in a proximate direction (toward the inlet 212). This allows for the liquid to drain from the first housing 202 in to the second housing 204. Openings spaced around the bottom of the projection 350 allow the liquid to pass around the projection 350 and along the inner surface 364. Since the proximal end 340 of the second housing 204 is, by way of the latch 334, removably mounted to the first housing 202, removing the second housing 204 also removes the projection 350 from the drain end 304, allowing the sealing member 302 to seal the outlet 324, even if the second magnet 280 moves to allow the liquid to pass through the opening 246. This allows the patient to, at least temporarily, be separated from a collection bag, as illustrated in FIG. 10.

As noted above, the first and second housings 202,204 are removably attached to one another by the latch 334 as noted above. By “removably attached,” Applicant means that the two housings 202,204 are intended to and can repeatedly engage and disengage one another without any other elements (e.g., glues, adhesives, bands, etc.), structures, or destroying any portions or parts that are intended to be used to attach housings 202,204. As best seen in FIGS. 13 and 14, the latch 334 is integral with the button 360, which when pressed then causes the latch 334 to be disengaged from the outer portion 330 of the drain end 304, and the housing 204 can be removed from housing 202. It should also be noted that due to the tight fit of the cylindrical center portion 320 in the inner opening 344, only one latch 334 needs to be used to maintain the connection between the two housings 202,204. Other types of latches and numbers of latches may also be used with the valve 200 and still come within the scope of the present invention.

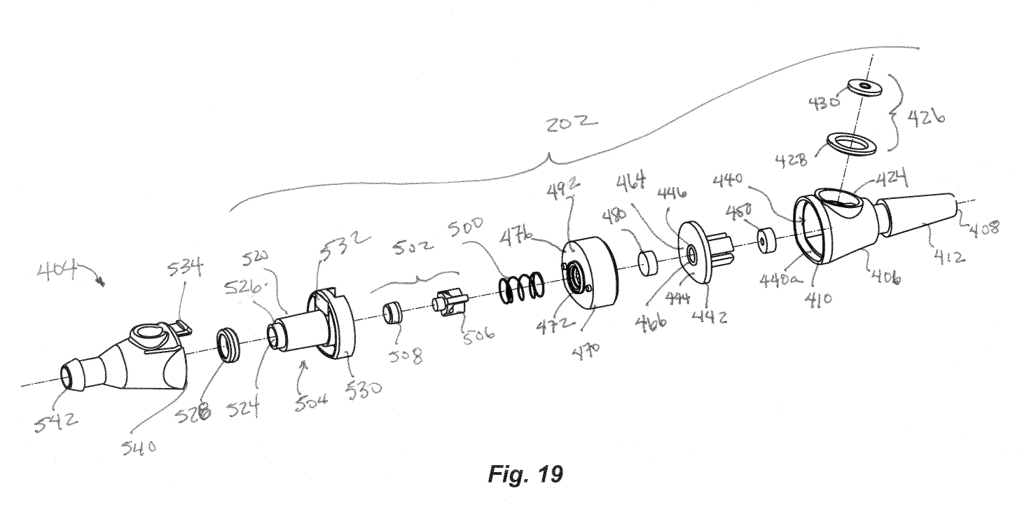

Another embodiment of a valve 400 according to the present invention is illustrated in FIGS. 19-25. The valve 400 has a first housing 402 and a second housing 404 that is removably attachable to the first housing 402. The first housing 402 has a catheter connector 406 having a proximal end 408 and a distal end 410, the proximal end 408 of the catheter connector 406 has an inlet 412 that is configured to engage a tubing (see, e.g., FIG. 10). The inlet 412, as illustrated, has a generally smooth outer surface that increases in diameter from the end of the inlet 412 toward the remaining portion of the catheter connector 406. The inlet 412 may have any other configuration that allows for connection to tubing and still fall within the scope of the present invention. The first and second housings 402,404 are preferably made from a polycarbonate.

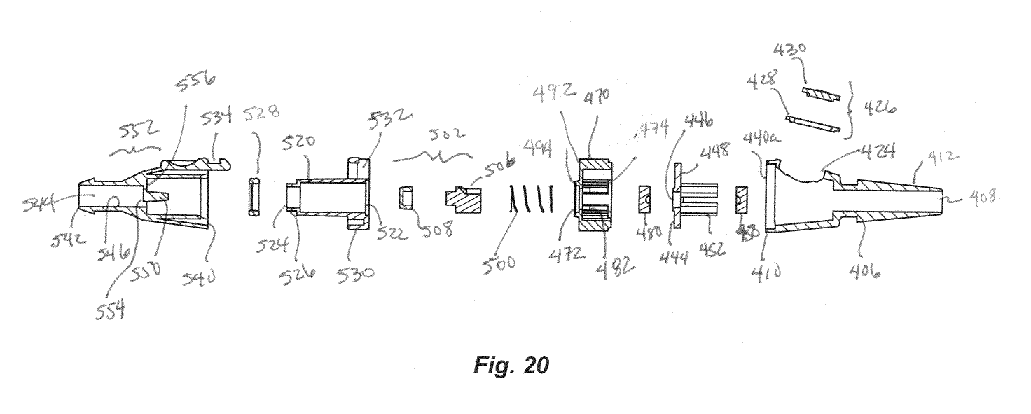

As best illustrated in FIGS. 19 and 20, the catheter connector 406 also has an opening 424 that functions as a sampling port. The opening 424 preferably has a needleless port 426. The needleless port 426 allows for insertion of a needleless syringe (not shown) to withdraw a sample of the liquid (generally urine) for testing. The needleless port 426 is preferably a resealable opening. The needleless port 426 has a main body 428 and the resealable portion 430, the resealable portion 430 may or may not have a pre-cut slit therein. It is also possible that the needleless port 426 is a single unit and not made of two different portions. The main body 428 is preferably made of polypropylene and the resealable portion 430 is preferably made of Dynaflex® thermoplastic elastomer (available from GLS Corp.), but any appropriate materials may be used. Additionally, a syringe with a needle may also be used with the needleless port 426, as discussed in more detail below regarding the configuration of the needleless port 426 to the internal structures.

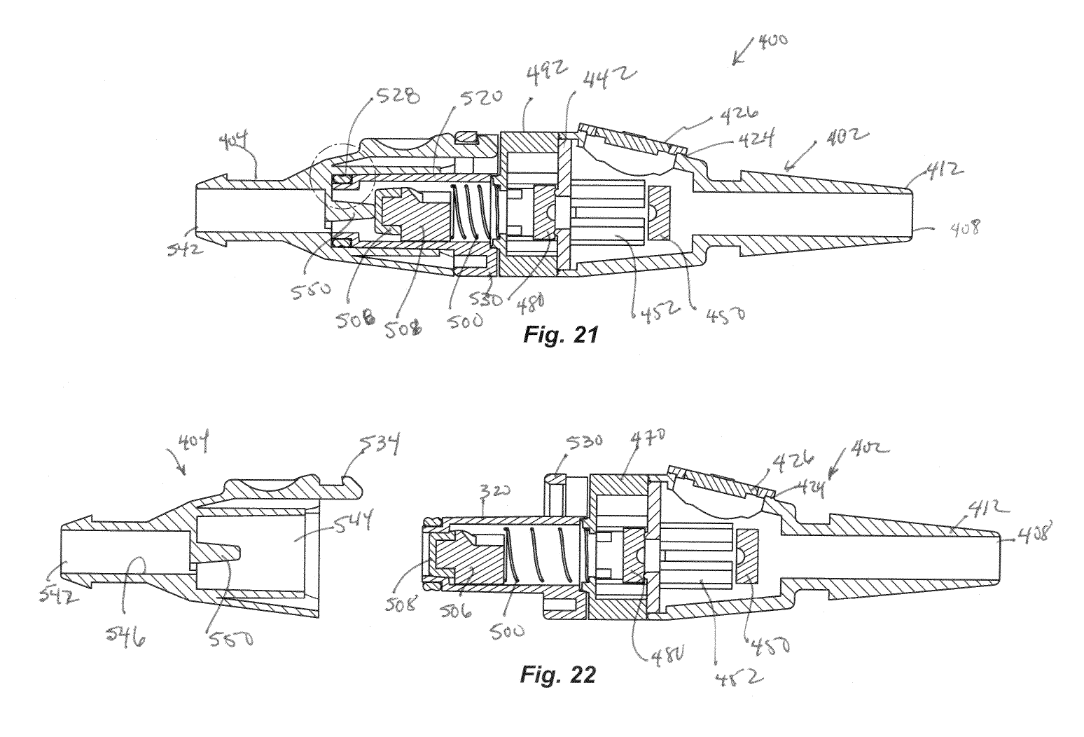

The catheter connector 406 also has an opening 440 at the distal end 420 that is in fluid communication with the inlet 412. The opening 440 has a recessed portion 440a adjacent the distal end 410 into which a first magnet housing 442 is disposed, sealing the opening 440 of the catheter connector 406. See, e.g., FIGS. 21 and 22. The first magnet housing 442 is preferably a monolithic element that is injection molded as a single element from Dynaflex® thermoplastic elastomer. However, the various parts of the first magnetic housing 442 may also be assembled and attached to one another by appropriate means for the materials (e.g., adhesives, ultrasonic welding, etc.). As best seen in FIG. 20, first magnetic housing 442 has a base plate 444 that has the same configuration as the opening 440 and an opening 446 in the base plate 444 to allow the liquid to pass therethrough. On a first side 448 of the base plate 444 a first magnet 450 is secured. The first magnet 450 is secured in between extensions 452. The extensions 452 extend from the first side 448 of the base plate 444, and keep the first magnet 450 in a predetermined relationship with the opening 446 and a second magnet, which is described in more detail below.

The first magnet housing 442 also preferably has on a second side 464 a raised portion 466 around the opening 446. As described in more detail below, the raised portion 466 provides a surface against which the second magnet can maintain contact to seal the opening 446. The extensions 452 are configured the same as extensions 52, discussed above. Similarly, the first magnet housing 442 may be same as first magnet housing 242 and still fall within the scope of the claimed invention.

As best illustrated in FIGS. 19-22, second magnet housing 470, also a part of the first housing 402, is attached to the first magnet housing 442 and the catheter connector 406 and extends distally from the catheter connector 406. The second magnet housing 470 is preferably generally cup-shaped with a central opening 472 to allow the liquid to flow therethrough. The central opening 472 has extensions 474 that extend from the distal end 476 toward the catheter connector 406 and the first magnet housing 440 that slidingly hold a the second magnet 480. Preferably, there are three extensions 474, but there may be other numbers of extensions and still be within the scope of the invention. The extensions 474 also have a surface 482 to engage the second magnet 480 and prevent the second magnet 480 from moving too far distally (toward the distal end 476). The second magnet 480 is drawn magnetically toward the first magnet 450 causing the second magnet 480 to engage the raised portion 466 around the opening 446, thereby closing the opening 446 and preventing the flow of liquid through the valve 400. However, when sufficient liquid is present in the opening 440 and exerts pressure on the second magnet 480 sufficient to overcome the magnetic attraction between the two magnets 450,480, then the second magnet 480 moves (is pushed) axially away from the opening 446 within the extensions 474 (but no farther than the surfaces 482) to allow the liquid to drain through the opening 446 (and the tubing that is inserted into bladder of a patient). When the liquid has drained away and removes this force, then the magnetic attraction causes the second magnet 480 to once again close the opening 446.

The second magnet housing 470 has on a bottom side 492 an elevated ring portion 494 that engages and centrally maintains an elastic member 500, which in turn, engages and biases a sealing member 502 toward the second housing 404 in a drain end 504. While a coil spring is illustrated as the elastic member 500, any appropriate style of spring or elastic member may be used to bias the sealing member 502. The sealing member 502 preferably has two elements, a main sealing member 506 and a resilient cover member 508 that is attached to the main sealing member 506. However, the sealing member 502 may be one integral element rather than two separate elements and may be made from a single material rather than multiple materials.

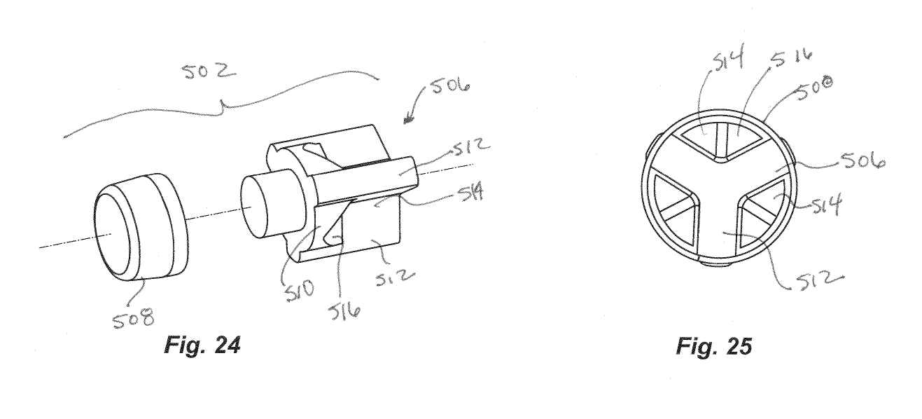

Turing to FIGS. 24 and 25, the main sealing member 506 has a bottom portion 510 that is generally cylindrical and has three portions 512 extending slightly outward from the bottom portion 510 and then away from the bottom portion 510 in a proximal direction toward the elastic member 500. The three upward extending portions 512, having a generally three-pointed-star-like configuration, support the distal end of the elastic member 500. See FIG. 25. In a middle portion of the main sealing member 506, the generally cylindrical portion is tapers back to toward the center of the main sealing member 506, leaving only the three upward extending portions 512, with openings 514 between the three upward extending portions 512 at the top where it engages the elastic member 500. As can be seen in FIG. 25, the liquid, as it would flow downward into the figure, will follow the openings 514 between each of the upward extending portions 512, then be diverted by the sloped portions 516 formed from cylindrical portion that tapers back to force the liquid toward the outward edge of the main sealing member and then down the sides of the bottom portion 510. Thus, even if the elastic member 500 does not allow the liquid to flow out between the individual coils or the elastic member otherwise prevents the flow of liquid therethrough, the liquid can drain out through the openings 514 through the main sealing member 506.

The drain end 504, which constitutes the last element of the first housing 402, is attached to the second magnet housing 470 and is in fluid communication with the inlet 412 in the catheter connector 406. See FIGS. 21-22. The drain end 504 has a generally cylindrical center portion 520 in which the sealing member 502 is slidingly movable. The cylindrical center portion 520 has an inlet 522 and an outlet 524. The sealing member 502 is, as noted above, biased away from the second magnet housing 470 and toward the outlet 524 of the drain end 504. The drain end 504 also has an outer portion 530 that is secured to the bottom side 492 of the second magnet housing 470. Between the cylindrical center portion 520 and the outer portion 530 is an opening 532 into which a cantilevered latch 534 from the second housing 404 is inserted to hold the second housing 404 to the first housing 402.

The cylindrical center portion 520 has a groove 526 near the outlet 524 and preferably adjacent the outlet 524. The location of the groove 526 need only be such that an elastic element 528, such as a double o-ring, can engage the second housing 404 as described in more detail below.

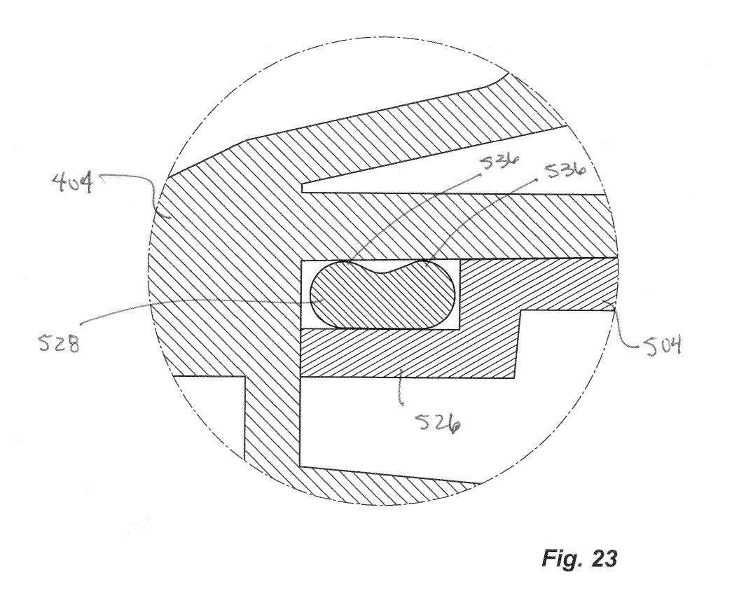

While the figures illustrate that the elastic element 528 is placed over the end of cylindrical center portion 520 at the groove 526, it is preferred that the elastic element 526 is injection molded on to the cylindrical center portion 520 at the groove 526. The elastic element 528 is illustrated in more detail in FIG. 23. Elastic element 528 is illustrated as a single element with two radial protrusions 536,538 that extend radially around the elastic element 528. The radial protrusions 536,538 are preferably rounded and extend around the circumference of the cylindrical center portion 520. However, two individual elastic elements would also be within the scope of the present invention. The two radial protrusions 536,538 are preferably of different diameters, in that the radial protrusion 536 is slightly smaller than radial protrusion 538 by about several hundred of a thousandths of an inch. The two radial protrusions 536,538 are preferably different sizes since the opening in the second housing is slightly narrowing as the cylindrical center portion 520 is inserted, to ensure a tight fit and eliminate any leakage.

The second housing 404 has a proximal end 540 and a distal end 542. The second housing 404 has an inner opening 544 that extends between the proximal end 540 and the distal end 542 and defines an inner surface 546.

Positioned within the inner opening 544 is a projection 550 that extends from central portion 552 of the inner opening 544 toward the proximal end 540. The inner opening 544 also has in the central portion 552 (and preferably at the location of the distal end 554 of the projection 550) a proximally-facing surface 556. The inner opening 544 is configured and sized to receive the cylindrical center portion 520 of the first housing 402 therein and the proximally-facing surface 556 is positioned and configured such that the outer portions of outlet 524 of the drain end 504 make contact with the proximally-facing surface 556 (and in particular the elastic element 526 in the groove 522) and seal the valve 400 to prevent leaking when the two housings 402,404 are attached. The projection 550 is configured and sized, when the second housing 404 is connected to the first housing 402, to engage the sealing member 502 through the outlet 524 of the drain end 504 and move it axially and in a proximate direction (toward the inlet 412). See FIG. 21. This allows for the liquid to drain from the first housing 402 in to the second housing 404. Openings spaced around the bottom of the projection 550 allow the liquid to pass around the projection 550 and along the inner surface 564. Since the proximal end 540 of the second housing 404 is, by way of the latch 534, removably mounted to the first housing 402, removing the second housing 404 also removes the projection 550 from the drain end 504, allowing the sealing member 502 to seal the outlet 524, even if the second magnet 480 moves to allow the liquid to pass through the opening 446. This allows the patient to, at least temporarily, be separated from a collection bag, as illustrated in FIG. 10.

As noted above, the first and second housings 402,404 are removably attached to one another by the latch 534 as noted above. See FIG. 22. By “removably attached,” Applicant means that the two housings 402,404 are intended to and can repeatedly engage and disengage one another without any other elements (e.g., glues, adhesives, bands, etc.), structures, or destroying any portions or parts that are intended to be used to attach housings 402,404. As best seen in FIGS. 20-22, the latch 534 is integral with the button 560, which when pressed then causes the latch 534 to be disengaged from the outer portion 530 of the drain end 504, and the housing 404 can be removed from housing 402. It should also be noted that due to the tight fit of the cylindrical center portion 520 in the inner opening 544, only one latch 534 needs to be used to maintain the connection between the two housings 402,404. Other types of latches and numbers of latches may also be used with the valve 400 and still come within the scope of the present invention.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

I claim:

1. A valve for regulating the flow of a liquid therethrough comprising:
a first housing having an inlet and an outlet;
a second housing removably attachable to the first housing, the second housing having an opening extending therethrough; and
a sealing member disposed in the first housing adjacent the outlet, the sealing member having at least one opening between a top end and the bottom end of the sealing member to allow the liquid to flow therethrough, the sealing member also sealing the outlet in the first housing when the second housing is disengaged from the first housing.

2. The valve according to claim 1, wherein the outlet of the first housing engages the opening in the second housing.

3. The valve according to claim 1, wherein the first housing has an outer surface, the outer surface having a groove adjacent the outlet and an elastic member disposed within the groove, the elastic member to engage an inner surface of the opening of the second housing when attached thereto.

4. The valve according to claim 3, wherein the elastic member has two radial protrusions that extend around the elastic member, one of the two radial protrusions having a diameter that is smaller than the other of two radial protrusions.
